(19) United States
(12) Patent Application Publication
(10) Pub. No.: US 2025/0127967 A1
(43) Pub. Date: Apr. 24, 2025

(54) BREAST PUMP BODY AND BREAST PUMP

(71) Applicant: FIMILLA (SHANGHAI) MATERNITY & BABY ARTICLES CO., LTD., Shanghai (CN)

(72) Inventor: Keyong CHENG, Shanghai (CN)

(73) Assignee: FIMILLA (SHANGHAI) MATERNITY & BABY ARTICLES CO., LTD., Shanghai (CN)

(21) Appl. No.: 18/892,604

(22) Filed: Sep. 23, 2024

(30) Foreign Application Priority Data
Oct. 19, 2023 (CN) 202311361803.5

(51) Int. Cl. A61M 1/06

(52) U.S. Cl. CPC A61M 1/069; A61M 2205/07; A61M 2205/3331; A61M 2205/3653; A61M 2205/8206; A61M 2209/088

(57) ABSTRACT

A pump body of a breast pump includes an air pump, a battery, a valve, and a neck-hanging structure. A first accommodation housing and a second accommodation housing are provided at two ends of the neck-hanging structure respectively. One of the air pump and the battery is provided in the first accommodation housing, and the other of the air pump and the battery is provided in the second accommodation housing. At least one of the first accommodation housing and the second accommodation housing is provided with an air vent for communicating with the air pump. In the above pump body of the breast pump, the neck-hanging structure is provided, and the first accommodation housing and the second accommodation housing are provided at two ends of the neck-hanging structure. The pump body is supported by the neck-hanging structure, which frees hands and facilitates carrying.

### CROSS REFERENCE TO RELATED APPLICATIONS

This application claims the priority to Chinese patent application No. 202311361803.5, titled “BREAST PUMP BODY AND BREAST PUMP”, filed with the China National Intellectual Property Administration on Oct. 19, 2023, the entire disclosure of which is hereby incorporated by reference.

### FIELD

The present application relates to the technical field of maternal and child products, and in particular to a breast pump body and a breast pump having the breast pump body.

### BACKGROUND

There are a wide variety of breast pumps on the market. For instance, electric breast pumps can be subdivided into two types, i.e., unilateral electric breast pumps or bilateral electric breast pumps. A unilateral electric breast pump generally has a control assembly directly provided on top of a breast shield, and the control assembly includes an air pump, a solenoid valve, a control board, and so on. At least a one-way valve and a storage bottle are provided below the breast shield. The disadvantage of the unilateral electric breast pump is that it has to be held by hand during use. In addition, the unilateral electric breast pump is used to finish extracting milk at one breast before the other. A bilateral electric breast pump is equipped with two sets of breast shields and storage bottles as accessories, and further equipped with several silicon tubes and tube interfaces for connection.

The biggest disadvantage of these breast pumps is not hands-free during use. Even if a wearable T-shaped breast pump is used, a pump body needs to be carried with a user, or be placed on a table or attached around the user's waist, thereby interfering with routine movement and housework. The wearable T-shaped breast pump, the pump body and a T-joint are connected together, which causes an increased weight and volume. Therefore, a bra can hardly secure the breast pump in place and the breast pump is easy to deviate from an original place, hence causing a seal failure, and the user cannot move normally during extracting milk.

In summary, a problem to be solved by those skilled in the art presently is how to effectively solve the problem of inconvenient carrying of the pump body of the breast pump during breast pumping.

### SUMMARY

In view of the above, a first object of the present application is to provide a pump body of a breast pump, which is able to effectively solve the problem of inconvenient carrying of the pump body of the breast pump during operation. A second object of the present application is to provide a breast pump including the above pump body.

In order to achieve the above first object, the following technical solutions are provided according to the present application.

A pump body of a breast pump includes at least one air pump, at least one battery, a valve, and a neck-hanging structure, and the neck-hanging structure are provided with a first accommodation housing and a second accommodation housing at two ends of the neck-hanging structure respectively. One part of the at least one air pump and the at least one battery is provided in the first accommodation housing, and the other part of the at least one air pump and the at least one battery is provided in the second accommodation housing. At least one of the neck-hanging structure, the first accommodation housing, and the second accommodation housing is provided with an air vent for communicating with the air pump.

When the pump body of the breast pump is used, the pump body of the breast pump is hanged on a neck of a user by the neck-hanging structure. In this situation, the neck-hanging structure hangs around the neck, so that two ends of the neck-hanging structure are placed at a front side of the body. Correspondingly, the first accommodation housing and the second accommodation housing are respectively placed at the front side of the body and server as counterweights, so as to ensure steady of the neck-hanging structure. In a case that the air vent is communicated with a breast milk collector and the breast milk collector is properly placed, an extracting milk process may be started after the air pump is turned on. In the pump body of the breast pump, the neck-hanging structure is provided, and the first accommodation housing and the second accommodation housing are provided at two ends of the neck-hanging structure respectively. One of the air pump and the battery is provided at the first accommodation housing, and the other of the air pump and the battery is provided at the second accommodation housing, so that uniform forces are applied on the two ends, therefore the neck-hanging structure is stable. With the neck-hanging structure, the pump body is supported and hands are free, which is also conducive for carrying with. To sum up, the pump body of the breast pump can effectively solve the problem of inconvenient carrying of the pump body of the breast pump during breast pumping.

In some embodiments, at least two air vents are provided. One of the two air vents is a first air vent exposed and provided at the first accommodation housing, and the other one of the two air vents is a second air vent exposed and provided at the second accommodation housing.

In some embodiments, the first air vent and the second air vent can communicate with the same air pump. The air pump is provided in the first accommodation housing, and the battery is provided in the second accommodation housing. The battery can power the air pump through a conductor wire arranged along the neck-hanging structure, and the air pump can communicate to the second air vent through a tube arranged along the neck-hanging structure.

In some embodiments, the first accommodation housing is provided with a first switch valve therein to be connected between the air pump and the first air vent, and the second accommodation housing is provided with a second switch valve therein to be connected between the air pump and the second air vent.

In some embodiments, the pump body of the breast pump further includes a switch valve. One end of the switch valve can communicate with the air pump, and the other end of the switch valve can communicate with the first air vent and the second air vent through a T-joint.

In some embodiments, the two ends of the neck-hanging structure are each fixedly connected to the first accommodation housing or the second accommodation housing by a housing portion, causing a C shape formed by integration.

In some embodiments, an outer shell of the neck-hanging structure is fixedly connected with another outer shell of the first accommodation housing and yet another outer shell of the second accommodation housing, respectively. Inner sides of the neck-hanging structure, the first accommodation housing, and the second accommodation housing are all provided with a skin-friendly layer.

In some embodiments, a heating structure is provided at a middle part of the neck-hanging structure. The heating structure is powered by the battery for heating a back side of a neck.

In some embodiments, a micro switch is provided at least one of the air vents for detecting whether the tube is inserted.

In some embodiments, a pressure sensor and a controller are further included. The pressure sensor is configured to obtain an air pressure at the air vent and the controller is configured to control the air pump to stop if a detected value of the pressure sensor exceeds a preset value.

In order to achieve the above second object, a breast pump is provided according to the present application. The breast pump includes the pump body of the breast pump according to anyone of the above embodiments, and further includes a breast milk collector. An air vent of the pump body of the breast pump can communicate with the breast milk collector through a tube. Since the pump body of the breast pump can achieve the above technical effects, the breast pump including the pump body of the breast pump can achieve corresponding technical effects.

### BRIEF DESCRIPTION OF THE DRAWINGS

For more clearly illustrating embodiments of the present application or technical solutions in the conventional technology, the drawings referred to for describing the embodiments or the conventional technology will be briefly described hereinafter. Apparently, the drawings in the following description are only some examples of the present application, and for those skilled in the art, other drawings may be obtained based on the provided drawing without any creative efforts.

FIG. 1 is a schematic structural diagram showing a pump body of a breast pump according to an embodiment of the present application;

FIG. 2 is a schematic exploded view showing structure of the pump body of the breast pump according to an embodiment of the present application;

FIG. 3 is a schematic diagram showing wearing state of the pump body of the breast pump according to an embodiment of the present application;

### BRIEF DESCRIPTION OF DRAWINGS

| | | | |
|---|---|---|---|
| 1 | air pump, | 2 | battery, |
| 3 | neck, | 4 | neck-hanging structure, |
| 5 | first accommodation housing, | 6 | second accommodation housing, |
| 7 | first air vent, | 8 | second air vent, |
| 9 | first switch valve, | 10 | second switch valve, |
| 11 | heating structure, | 12 | pressure sensor, |
| 13 | micro switch, | 14 | skin-friendly layer, |
| 15 | outer shell, | 16 | tube and conductor wire, |
| 17 | inner support, | 18 | controller, |
| 19 | multidimensional guide button. | | |

### DETAILED DESCRIPTION

Embodiments of the present application disclose a pump body of a breast pump, which can effectively solve the problem of inconvenient carrying of the pump body of the breast pump during extracting milk.

The technical solutions according to the embodiments of the present application will be described clearly and completely as follows in conjunction with the drawings in the embodiments of the present application. It is apparent that the described embodiments are only some of the embodiments according to the present application, rather than all the embodiments. Any other embodiments obtained by those skilled in the art based on the embodiments in the present application without any creative work fall in the scope of protection of the present application.

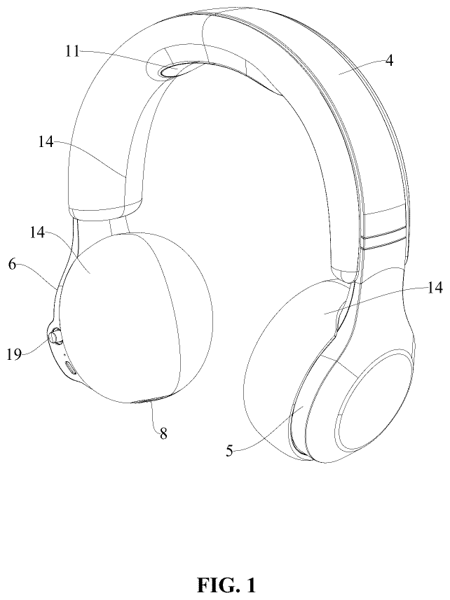

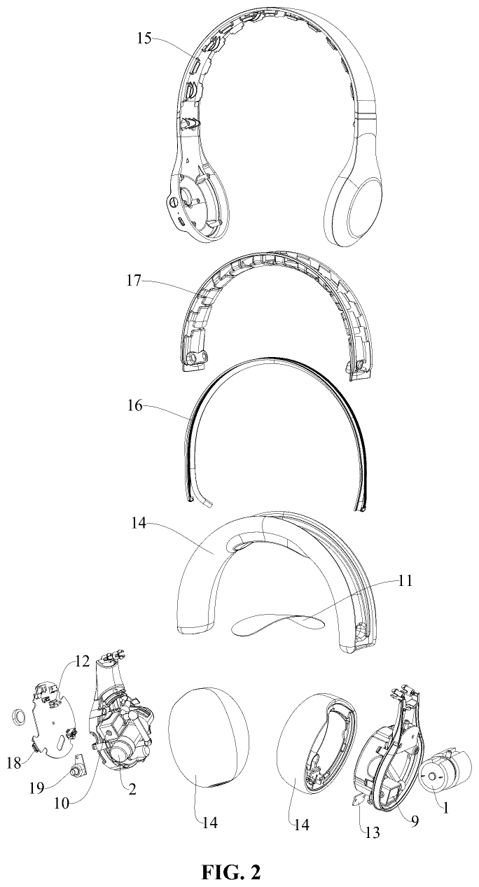

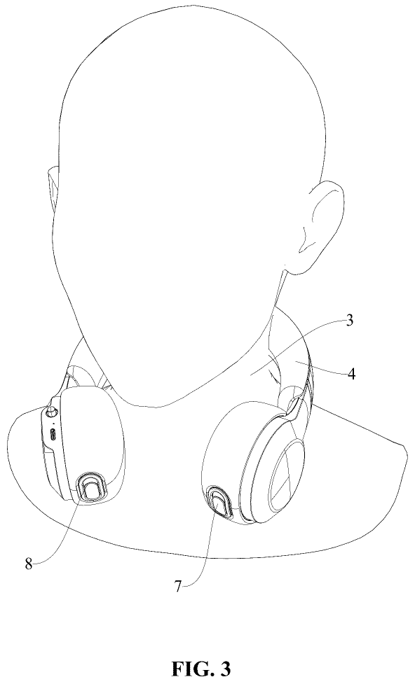

Referring to FIG. 1 to FIG. 3, FIG. 1 is a schematic structural diagram showing a pump body of a breast pump according to an embodiment of the present application, FIG. 2 is a schematic exploded view showing structure of the pump body of the breast pump according to an embodiment of the present application, and FIG. 3 is a schematic diagram showing wearing state of the pump body of the breast pump according to an embodiment of the present application.

In a specific embodiment, a pump body of a breast pump is provided. The pump body of the breast pump is configured to generate a negative pressure on a breast milk collector, causing the breast milk collector start to work. The breast milk collector fits over a breast. Specifically, the pump body of the breast pump mainly includes a first accommodation housing 5, a second accommodation housing 6, an air pump 1, a battery 2, a valve, and a neck-hanging structure 4.

The first accommodation housing 5 and the second accommodation housing 6 are respectively arranged at two ends of that neck-hanging structure 4, and may have the same size, where the same size refers to same or substantially same in external dimensions and/or weight, which aims to make forces applied on both ends of the neck-hanging structure 4 nearly equal, so as to effectively avoid generating a force in a left-right direction against a body of a user where the neck-hanging structure 4 contacts with the body. Inner cavities of both the first accommodation housing 5 and the second accommodation housing 6 need to accommodate certain parts, so overall weights thereof should be ensured to be equal or nearly equal with a weight difference no more than, for example, 10% of weight of a heavier one.

During use, the neck-hanging structure 4 may be placed over neck 3, so that the two ends thereof is placed at front side of the body, i.e., the first accommodation housing 5 and the second accommodation housing 6 are both placed at the front side of the body. Therefore, the whole pump body of the breast pump hangs on the neck 3 of the body and is supported by the neck 3 of the body. The neck-hanging structure 4 may be a band structure or a collar structure, where the collar structure may be semi-enclosed structure or a ring structure. In order to facilitate wearing, the neck-hanging structure 4 is basically the semi-enclosed structure, which is similar to a middle connection part of a headphone, while the first accommodation housing 5 and the second accommodation housing 6 are similar to ear cups of a headphone.

In some embodiments, to equalize weight, for the at least one air pump 1 and the at least one battery 2, some of them are provided in the first accommodation housing 5, and the other of them are provided in the second accommodation housing 6. Thus, overall weights of the first accommodation housing 5 and the second accommodation housing 6 may be close to each other, and of course, they can be equal to each other.

It should be noted that the pump body of the breast pump may be provided with only one air pump 1 and only one battery 2. In this situation, one of the air pump 1 and the battery 2 is disposed in the first accommodation housing 5, and the other one of the air pump 1 and the battery 2 is disposed in the second accommodation housing 6. Two air pumps 1 and two batteries may be provided. In this situation, one air pump 1 and one battery 2 are disposed in the first accommodation housing 5, and the other one air pump 1 and the other one battery 2 are disposed in the second accommodation housing 6.

In some embodiments, at least one of the neck-hanging structure 4, the first accommodation housing 5, and the second accommodation housing 6 is provided with an air vent communicating with the air pump 1 for adjusting air pressure at the air vent, such as pumping or supplying air, by the air pump 1. Specifically, the pump body of the breast pump may be provided with only one air vent at the first accommodation housing 5 or the second accommodation housing 6. Alternatively, the pump body of the breast pump may be provided with only two air vents at the first accommodation housing 5 or the second accommodation housing 6, or one of the air vents is provided at the first accommodation housing 5 and the other one of the air vents is provided at the second accommodation housing 6. Alternatively, no less than three air vents may be provided. The air vents are configured to communicate with the breast milk collector so as to make the breast milk collector perform pumping. The air vents provided at the neck-hanging structure 4 may be provided near the first accommodation housing 5, or may be provided near the second accommodation housing 6.

In some embodiments, when the pump body of the breast pump is used, the whole pump body of the breast pump hangs on the neck 3 of the body by the neck-hanging structure 4. In this situation, the neck-hanging structure 4 hangs around the back side of the neck 3, so that the two ends of the neck-hanging structure 4 are placed at the front side of the body. Correspondingly, the first accommodation housing 5 and the second accommodation housing 6 are respectively placed at the front side of the body and server as counterweights, so as to ensure steady of the neck-hanging structure. In a case that the air vent is communicated with the breast milk collector and the breast milk collector is properly placed, an extracting milk process may be started after the air pump 1 is turned on. In the pump body of the breast pump, the neck-hanging structure 4 is provided, and the first accommodation housing 5 and the second accommodation housing 6 are provided at two ends of the neck-hanging structure 4 respectively. One of the air pump 1 and the battery 2 is provided at the first accommodation housing 5, and the other of the air pump 1 and the battery 2 is provided at the second accommodation housing 6, respectively, so that forces applied on the two ends are almost equal, and therefore the neck-hanging structure is stable. With the neck-hanging structure, the pump body is supported and hands are free, which is also conducive for taking along. To sum up, the pump body of the breast pump can effectively solve the problem of inconvenient carrying of the pump body of the breast pump during extracting milk.

In some embodiments, considering that the air vent connects to the breast milk collector through a tube and gravity of the tube applies a pulling force on the air vent. In a case that there are at least two air vents, one of the two air vents is a first air vent 7 exposed and provided at the first accommodation housing 5, the other of the two air vents is a second air vent 8 exposed and provided at the second accommodation housing 6, so that forces applied on the first accommodation housing 5 and the second accommodation housing 6 are almost equal. At the same time, the first air vent 7 and the second air vent 8 are corresponding to a left breast milk collector and a right breast milk collector respectively, so that forces applied on the left and right breast milk collectors are almost equal, hence pumping efficiency is improved.

In some embodiments, as described above, for the structure having the first air vent 7 and the second air vent 8, the first accommodation housing 5 is provided with the air pump 1 and the battery 2 to be corresponding to the first air vent 7. Correspondingly, the second accommodation housing 6 is provided with the air pump 1 and the battery 2 to be corresponding to the second air vent 8. The above structure enables any one of the first accommodation housing 5 and the second accommodation housing 6 forms an independent pumping system.

In some embodiments, in view of a high cost of the air pump 1 and the battery 2, the first air vent 7 and the second air vent 8 are preferably communicate with the same air pump 1. Specifically, as shown in FIG. 2, the air pump 1 may be disposed in the first accommodation housing 5, and the battery 2 may be disposed in the second accommodation housing 6. The battery 2 can power the air pump 1 through a conductor wire arranged along the neck-hanging structure 4. The air pump 1 can communicate to the second air vent 8 through a tube arranged along the neck-hanging structure 4. The conductor wire and/or the tube may be embedded in a 10 slot of the neck-hanging structure 4, or may thread through the neck-hanging structure 4. The air pump 1 may communicate with the first air vent 7 through a tube.

In some embodiments, the air pump may concurrently communicate with the first air vent 7 and the second air vent 8, and therefore the breast milk collector corresponding to the first air vent 7 and the breast milk collector corresponding to the second air vent 8 may be simultaneously put into operation, which however, may cause inconvenience in operation and is disadvantageous for making selection according to different situations. A valve may be provided between the air pump 1, the first air vent 7, and the second air vent 8, such as a three-way directional valve. By this valve, that one of the first air vent 7 and the second air vent 8 may communicate with the air pump 1 or both the first air vent 7 and the second air vent 8 may communicate with the air pump 1 can be achieved.

In view of the above, in an embodiment, a first switch valve 9 is provided in the first accommodation housing 5 to be connected between the air pump 1 and the first air vent 7, and a second switch valve 10 is provided in the second accommodation housing 6 to be connected between the air pump 1 and the second air vent 8, so as to respectively control switching on/off of the first air vent 7 and the second air vent 8. Therefore, it is possible to realize bilateral or unilateral breast pumping to fulfill requirements in more situations. Moreover, since the first switch valve 9 and the second switch valve 10 are respectively disposed in the first accommodation housing 5 and the second accommodation housing 6, weights thereof are in a better balance and forces applied on the two ends of the neck-hanging structure 4 are equal or almost equal.

The first switch valve 9 and the second switch valve 10 may be manual valves or solenoid valves, and solenoid valves are preferable, so as to achieve a quick control by button. In order to facilitate control, a circuit board and a corresponding electronic structure are generally provided, which are divided into two groups according to weight. One group is provided in the first accommodation housing 5, and the other group is provided in the second accommodation housing 6. Alternatively, the two groups are both provided in the first accommodation housing 5 or the second accommodation housing 6 in order to balance weight difference between the first accommodation housing 5 and the second accommodation housing 6.

In an embodiment, only one switch valve is provided. One end of the switch valve communicates with the air pump 1 and the other end of the switch valve communicates with the first air vent 7 and the second air vent 8 through a T-joint.

In some embodiments, the two ends of the neck-hanging structure 4 are each fixedly connected to the first accommodation housing 5 or the second accommodation housing 6 by a housing portion, thus a C shape is formed by integration. The C-shaped structure provides a flexible opening to facilitate stretching and accommodating the neck 3, so that the whole structure can hang on the neck 3 steadily, hence reducing swaying.

Connection between the two ends of the neck-hanging structure 4 and the first accommodation housing 5 as well as the second accommodation housing 6 may be integrally formed, clamped, screwed, or other ways of fixed connection. The housing may be a whole outer shell or a partial housing.

In some embodiments, an outer shell 15 of the neck-hanging structure 4 may be fixedly connected with another outer shell 15 of the first accommodation housing 5 and yet another outer shell 15 of the second accommodation housing 6, respectively. The outer shell 15 refers to the side away from the neck 3, and therefore an outer side is facilitated to form an integrated structure.

In some embodiments, in order to ensure strength of the neck-hanging structure 4, an inner support 17 is provided at inner side of the outer shell 15 of the neck-hanging structure 4. A skin-friendly layer 14 is attached to an inner side of the inner support 17. A tube and a conductor wire 16 are provided between the inner support 17 and the outer shell 15 or provided between the inner support 17 and the skin-friendly layer 14. For the tube and the conductor wire of the tube and the conductor wire 16, the at least one tube is a tube between the air pump 1 and the corresponding air vent, and at least one conductor wire is a conductor wire between the air pump 1 and the battery 2.

In some embodiments, inner sides of the neck-hanging structure 4, the first accommodation housing 5, and the second accommodation housing 6 are all provided with the skin-friendly layer 14 to enhance comfort in contact with a human body, especially when squeezed. The skin-friendly layer 14 is generally a soft layer, such as a soft leather layer, a soft fabric layer, or a soft sponge layer.

In some embodiments, a heating structure 11 is provided at middle part of the neck-hanging structure 4. The heating structure 11 is powered by the battery 2 and is configured to heat a back side of the neck 3, so as to relieve discomfort of the neck 3 and enhance wearing comfort during use. The heating structure 11 may be powered by the battery 2. The heating structure 11 is mainly a heating sheet, and a heating temperature thereof should not be too high with an aim of providing a warming effect. With the skin-friendly layer 14, the heating structure 11 may act on the back of the neck 3 of the body, so as to increase the heating effect. The heating structure 11 may use NTC (Negative Temperature Coefficient) for precise control and regulation of temperature, where the NTC refers to thermistor phenomena and material with negative temperature coefficient, whose resistance decreases exponentially with increasing temperature. PWM (Pulse Width Modulation) technology controls temperature range of PI (Polyimide) film to achieve a heating effect.

In some embodiments, a micro switch 13 is provided at the air vents to detect whether a tube is inserted, so as to keep the corresponding switch valve closed if no tube is inserted. In an embodiment, a first micro switch is provided at the first air vent 7, and the second micro switch is provided at the second air vent 8. If the first micro switch fails to detect a tube inserted into the first air vent 7, the first switch valve 9 keeps closed. The first switch valve 9 opens only if the first micro switch detects that the tube is inserted into the first air vent 7 and other conditions are met. If the second micro switch fails to detect a tube inserted into the second air vent 8, the second switch valve 10 keeps closed. The second switch valve 10 opens only if the second micro switch detects that the tube is inserted into the second air vent 8 and other conditions are met. Therefore, the breast pump is able to realize unilateral or bilateral breast pumping with a connection detection performed by the micro switch 13 triggered by insertion of the tube.

In some embodiments, a pressure sensor 12 and a controller 18 are further provided, where the pressure sensor 12 is configured to obtain an air pressure at air vent and the controller 18 is configured to control the air pump 1 to stop if a detected value of the pressure sensor 12 exceeds a preset value. Through the control by the controller 18 and the detection by the pressure sensor 12, accuracy of pumping force is improved. The air pump 1 is controlled by the detected value of the pressure sensor 12 correspondingly, which helps to improve accuracy of the negative pressure and adaptively adjust pumping strength and frequency.

In some embodiments, in order to facilitate input of corresponding instructions to the controller 18, the first accommodation housing 5 and/or the second accommodation housing 6 may be provided with a multidimensional guide button 19, such as an OSD (On Screen Display) five-dimension guide button, where the OSD refers to a screen display technology for displaying characters, graphics, and images on a display terminal. The multidimensional guide button 19 is configured to determine a button direction according to different voltage outputs, and therefore realize control. A MCU (Microcontroller Unit) is configured to determine power on/off, mode, shift up/down, heating, and perform control processing by a voice control panel, according to an electrical level of a five-direction button. An output of a negative pressure sensing operational amplifier circuit is used for making a determination during use. A motor stops if a preset pumping strength is reached, and a negative pressure is retained for pumping and emptying breast milk.

The present application further provides a breast pump based on the pump body of the breast pump disclosed in the above embodiments. The breast pump includes the pump body of the breast pump disclosed in any one of the embodiments hereinbefore, and includes a breast milk collector. An air vent of the pump body of the breast pump can communicate with the breast milk collector through a tube. Since the breast pump adopts the pump body of the breast pump provided in the above embodiments, the technical effect of the breast pump may refer to the above embodiments.

The embodiments in the specification are described in a progressive manner, and each embodiment focuses on the difference from other embodiments. Same and similar parts in each embodiment may be referred to each other.

According to the above description of the disclosed embodiments, those skilled in the art can implement or practice the present application. Various modifications to these embodiments are obvious to those skilled in the art, the general principles defined herein may be implemented in other embodiments without departing from the spirit and scope of the present application. Therefore, the present application is not limited to the embodiments described herein, but should be in accordance with the broadest scope consistent with the principle and novel features disclosed herein.

1. A pump body of a breast pump, comprising an air pump, a battery, a valve, and a neck-hanging structure, wherein
the neck-hanging structure is provided with a first accommodation housing and a second accommodation housing at two ends of the neck-hanging structure respectively;
one of the air pump and the battery is provided in the first accommodation housing, and the other of the air pump and the battery is provided in the second accommodation housing; and
at least one of the neck-hanging structure, the first accommodation housing, and the second accommodation housing is provided with an air vent for communicating with the air pump.

2. The pump body of the breast pump according to claim 1, wherein at least two air vents are provided;
one of the at least two air vents is a first air vent exposed and provided at the first accommodation housing, and the other one of the at least two air vents is a second air vent exposed and provided at the second accommodation housing.

3. The pump body of the breast pump according to claim 2, wherein the first air vent and the second air vent communicate with a same air pump;
the air pump is provided in the first accommodation housing, and the battery is provided in the second accommodation housing; and
the battery is configured to power the air pump through a conductor wire arranged along the neck-hanging structure, and the air pump is configured to communicate to the second air vent through a tube arranged along the neck-hanging structure.

4. The pump body of the breast pump according to claim 3, wherein a first switch valve is provided in the first accommodation housing to be connected between the air pump and the first air vent, and a second switch valve is provided in the second accommodation housing to be connected between the air pump and the second air vent.

5. The pump body of the breast pump according to claim 3, further comprising a switch valve, wherein one end of the switch valve is configured to communicate with the air pump, and the other end of the switch valve is configured to communicate with the first air vent and the second air vent through a T-joint.

6. The pump body of the breast pump according to claim 1, wherein the two ends of the neck-hanging structure are each fixedly connected to the first accommodation housing or the second accommodation housing by a housing portion, which forms a C shape by integration.

7. The pump body of the breast pump according to claim 6, an outer shell of the neck-hanging structure is fixedly connected with another outer shell of the first accommodation housing and yet another outer shell of the second accommodation housing, respectively; inner sides of the neck-hanging structure, the first accommodation housing, and the second accommodation housing are all provided with a skin-friendly layer.

8. The pump body of the breast pump according to claim 1, further comprising a heating structure provided at a middle part of the neck-hanging structure; the heating structure is powered by the battery for heating a back side of a neck.

9. The pump body of the breast pump according to claim 8, further comprising a micro switch provided at least one of the air vents, wherein the micro switch is configured to detect whether the tube is inserted.

10. The pump body of the breast pump according to claim 9, further comprising a pressure sensor and a controller, wherein the pressure sensor is configured to obtain an air pressure at the air vent and the controller is configured to control the air pump to stop if a detected value of the pressure sensor exceeds a preset value.

11. A breast pump, comprising a breast milk collector and a pump body of a breast pump according to claim 1, wherein an air vent of the pump body of the breast pump is configured to communicate with the breast milk collector through a tube.

12. The pump body of the breast pump according to claim 2, wherein the two ends of the neck-hanging structure are each fixedly connected to the first accommodation housing or the second accommodation housing by a housing portion, which forms a C shape by integration.

13. The pump body of the breast pump according to claim 3, wherein the two ends of the neck-hanging structure are each fixedly connected to the first accommodation housing or the second accommodation housing by a housing portion, which forms a C shape by integration.

14. The pump body of the breast pump according to claim 4, wherein the two ends of the neck-hanging structure are each fixedly connected to the first accommodation housing or the second accommodation housing by a housing portion, which forms a C shape by integration.

15. The pump body of the breast pump according to claim 2, further comprising a heating structure provided at a middle part of the neck-hanging structure; the heating structure is powered by the battery for heating a back side of a neck.

16. The pump body of the breast pump according to claim 3, further comprising a heating structure provided at a middle part of the neck-hanging structure; the heating structure is powered by the battery for heating a back side of a neck.

17. The pump body of the breast pump according to claim 4, further comprising a heating structure provided at a middle part of the neck-hanging structure; the heating structure is powered by the battery for heating a back side of a neck.

18. A breast pump, comprising a breast milk collector and a pump body of a breast pump according to claim 2, wherein an air vent of the pump body of the breast pump is configured to communicate with the breast milk collector through a tube.

19. A breast pump, comprising a breast milk collector and a pump body of a breast pump according to claim 3, wherein an air vent of the pump body of the breast pump is configured to communicate with the breast milk collector through a tube.

20. A breast pump, comprising a breast milk collector and a pump body of a breast pump according to claim 4, wherein an air vent of the pump body of the breast pump is configured to communicate with the breast milk collector through a tube.
